(19) United States
(12) Patent Application Publication
(10) Pub. No.: US 2019/0330585 A1
(43) Pub. Date: Oct. 31, 2019

(54) Bioreactors and Improvements in Their Heating

(71) Applicant: GE Healthcare (SV) Corp, Sunnyvale, CA (US)

(72) Inventor: Philip Vanek, Ashburn, VA (US)

(21) Appl. No.: 16/470,758

(22) PCT Filed: Dec. 20, 2017
(86) PCT No.: PCT/EP2017/083739

§ 371 Date: Jun. 18, 2019

Related U.S. Application Data
(60) Provisional application No. 62/436,825, filed on Dec. 20, 2016.

(51) Int. Cl. C12M 1/02; C12M 1/00

(52) U.S. Cl. CPC C12M 41/22; C12M 23/14; C12M 41/14; C12M 23/22; C12M 23/20

(57) ABSTRACT

Disclosed is a flexible bag bioreactor (100) having top and bottom walls wall (105,115) defining an internal culture volume (101). The top wall (105) at least the incorporates one or more electrically resistive heating elements (109 FIG. 2d). Also disclosed is a layered sheet heater (205) for bioreactors comprising, one or more layers (206,207) and (208) of flexible transparent polymer sheet, one or more electrically resistive heating elements (209) in or on said one or more polymer sheets, an optional layer of adhesive (210) on the, or on one of the polymer sheets, and an optional peelable backing layer over said adhesive layer, wherein either: said layered in sheet heater (205) is transparent over substantially its whole area; or wherein heating elements (209) are opaque but formed with gaps therebetween of sufficient width so as not to obscure, or completely obscure the majority (>50%) of light from propagating through the layers of the heater; or wherein the or each layer has a portion which is free from said heating elements, so as to provide said transparency in the element-free area.

### TECHNICAL FIELD

The present invention relates to bioreactors and improvements in bioreactor heating, particularly, electrical resistance heating.

### BACKGROUND

Various bioreactor systems are commercially available, which provide an enclosed volume for the culture of cells, microorganisms, and the like. All include some form of heating. Generally, stable and controllable heating is desirable to provide a satisfactory environment in which to culture the cells etc. Recent advances in immunotherapeutics has led to a need for bioreactors which are of lower volume, and desirably should be low cost and yet reliable, to provide affordable and dependable treatment. One way which that need has been addressed is to make much of the bioreactor system hardware around the bioreactor, including the heating parts, reusable, while making the bioreactor in the form of a flexible bag, which bag can be discarded once used. A rocking platform can be used instead of internal stirring means, which lowers the cost of the disposable part even further. One such system is disclosed in U.S. Pat. No. 6,544,788.

Conventionally, in commercial embodiments of that system heating parts are located in the rocking tray under the bag to heat the bag in use. That system is generally satisfactory, however, in the immunotherapeutic application mentioned above, for example in autologous immunotherapy techniques, seed cells are cultured in a small volume of liquid initially. Small volumes need very careful heat control, and fixed size reusable hardware is generally not optimized for the initial small volume. One problem is that condensation forms at the top of the bag and that can have undesirable effects. That condensation takes water from the culture liquid and thereby changes the pH and osmolality of the culture liquid. That change alters the culture conditions and can impact on the multiplication rate of cultured cells. In extreme conditions, cell death occurs. Given that seed cells from a donor for use in autologous therapies may be irreplaceable, it is an unacceptable risk to have significant condensation in a bioreactor, particularly where the volume of culture liquid is small, for example below about 1 litre, more particularly 500 ml or less.

Since few autologous seed cell batches behave in the same way, it is necessary to measure and adjust culture parameters—for example cell concentration, oxygen/CO₂ levels, and the supply of culture additives. It is convenient also to have sight of the cell culture through the bioreactor, so that a visual inspection can be made and so that light based measurements can be taken. For this purpose transparent or at least translucent bioreactors are preferred for smaller volumes. Herein, the word ‘transparent’ or similar terms relating to materials, are intended to encompass a range of light transmissivity from completely clear transparency, to translucent properties, where a majority of light propagates through a material albeit possibly in a diffuse or semi diffuse manner, including for example material which could be described as diaphanous. Providing an insulated bioreactor would reduce the problem of condensation, but would also eliminate or at least diminish the transparency of the bioreactor desirable for inspection/measurement.

### SUMMARY OF THE INVENTION

An object of the present invention is to provide a low cost, reduced condensation bioreactor which may be disposable.

The inventor has realized that that object can be addressed by providing a bioreactor having walls defining an internal culture volume, at least a portion of the walls including electrically resistive heating elements at least on or in a top portion of the bioreactor, which elements, in embodiments, are sufficiently narrow so as not to obscure, or completely obscure the culture volume, or have an area on the wall(s) where they are absent so that the culture volume of the bioreactor is visible. In addition, the inventor has realised that such elements, when made narrow enough to not obscure the view into the bioreactor, are flexible enough to be incorporated with a low cost flexible bioreactor bag, which can be made low cost and disposable.

Thereby, a bioreactor can be made which is low cost, so as to be disposable, for example in the form of a flexible bag formed substantially from polymer. The heating elements can be thin and flexible also, which allows a bag to flex, fold or bend, and does not interfere with the visibility of the contents of the bag.

According to an aspect of the invention, there is provided, a bioreactor having at least one wall defining an internal culture volume including a top wall which is intended to be at the top of the bioreactor in use, wherein at least the top wall incorporates one or more electrically resistive heating elements.

In an embodiment: the, or each wall is formed from a material which is transparent; the, or each heating element is sufficiently narrow so as not to obscure, or completely obscure, the culture volume; and/or the wall or walls which have heating elements have an area which includes heating element(s) and a further area where the element(s) are absent so that the culture volume of the bioreactor is visible.

In an embodiment, the bioreactor is formed from substantially a flexible bag-like bioreactor and the wall, or, one or more of said walls, is formed from a polymer material incorporating said element(s) in the respective wall(s) or on the surface of said wall(s)

According to another aspect of the invention, there is provided a layered sheet heater for bioreactors comprising, one or more layers of flexible transparent polymer sheet, one or more electrically resistive heating elements in or on said one or more polymer sheets, an optional layer of adhesive on the, or on one of the polymer sheets, and an optional peelable backing layer over said adhesive layer, wherein either: said layered sheet heater is transparent over substantially its whole area; or wherein heating elements are opaque but formed with gaps therebetween of sufficient width so as not to obscure, or not completely obscure the majority (>50%) of light from propagating through the layers of the heater; or wherein the or each layer has a portion which is free from said heating elements, so as to provide said transparency in the element-free area.

More aspects, advantages and benefits of the present invention will become readily apparent to the skilled person in view of the detailed description below, where features or elements of the same embodiment or described together are not necessarily intended to be encompassed by the ambit of a single claim, and therefore may be claimed separately without introducing new material.

### DRAWINGS

The invention will now be described in more detail with reference to the appended drawings, wherein:

FIG. 1 shows a bioreactor system;

FIG. 2a,b&c show different views a bioreactor incorporating a heating element;

FIG. 3a shows an exploded view of the bioreactor in FIG. 2;

FIG. 3b shows an alternative arrangement to the arrangement shown in FIG. 3a;

FIG. 4 shows a bioreactor heating sheet;

FIG. 5 shows an exploded view of heating sheet shown in FIG. 4.

### DETAILED DESCRIPTION

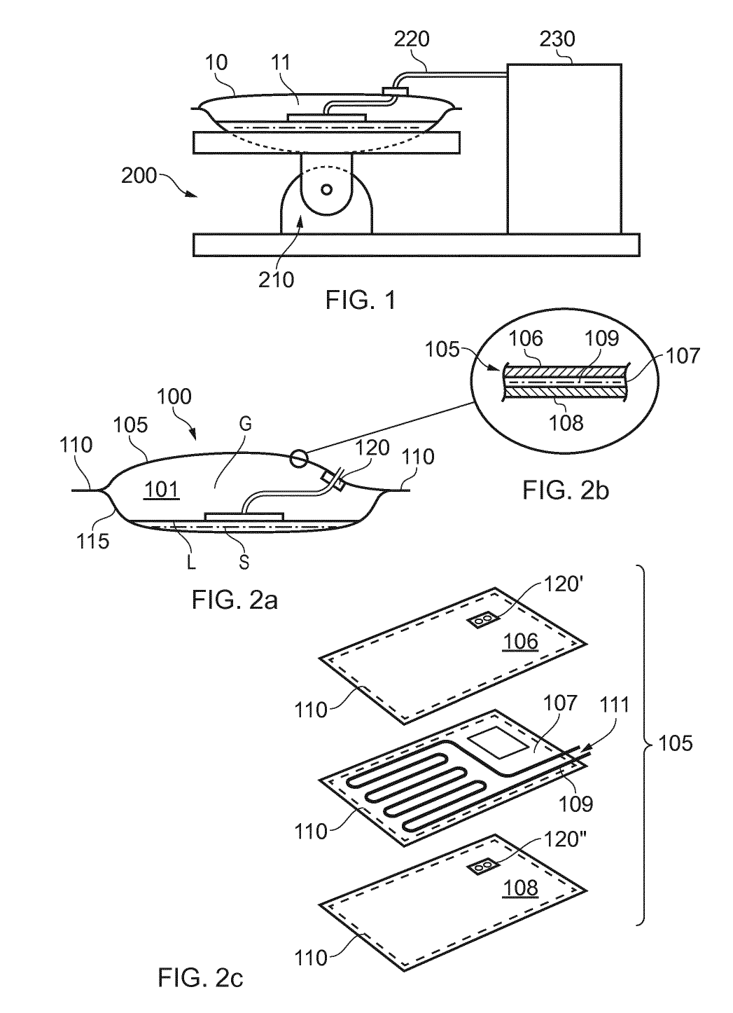

FIG. 1 illustrates a bioreactor 100 in this case in the form of a flexible bag of thermoplastic polymer sheet material defining a culture volume 11 in use. The bag 10 is used together with a bioreactor system 200, which system includes an electrically heated rocking tray 210 for supporting and agitating culture liquid in the bag 100 during cell culture. The system 200 provides a gas, liquid and signal inputs/outputs via conduits 220, and further includes a controller 230 for controlling the culture parameters, all of which is generally known, for example as available commercially under the trade name WAVE bioreactor system, by GE Healthcare, the features of such commercially available systems being incorporated herein by reference.

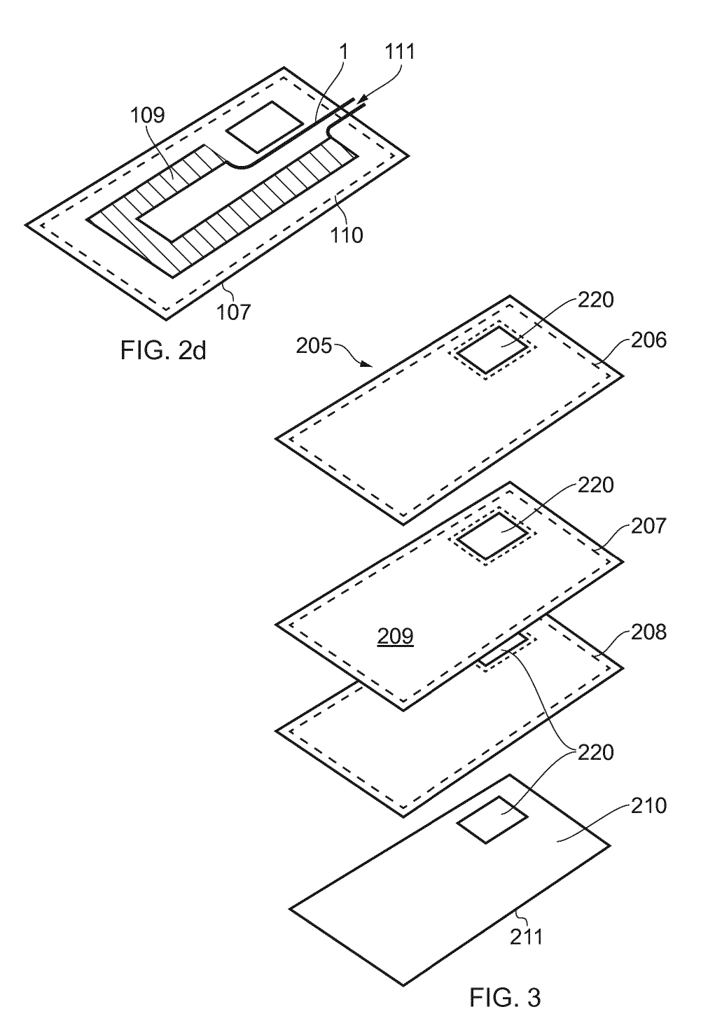

FIG. 2a shows a novel bioreactor 100 in section, which can be used together with the conventional bioreactor system illustrated in FIG. 1, in place of the bioreactor 10, with few modifications. As supplied the bioreactor 100 will be largely deflated bag, but is shown semi-inflated with a small liquid content L of about 50 ml and a gas head space G, both within its culture volume 101. The bag 100 provides a closed volume 101 for the culture of cells, or organisms, in a generally sterile environment. As mentioned above, it is the seed cells S in that initial liquid content L which are particularly vulnerable. This novel bag 100 is manufactured from a multiple sheet construction which at its edges has heat-fused joints 110 to form a pillow-like bag having an upper bioreactor wall 105 and a lower bioreactor wall 115. The skilled person could easily envisage other wall constructions. The upper wall 105 includes manifold block 120 which carries the gas/liquid input/output conduits 220 show in FIG. 1.

FIG. 2b shows an enlarged view of the upper wall 105, which has a layered construction, formed from an outer layer 106 of polyethylene sheet, and a middle, heating element carrier, layer 107 described in more detail below, which is sandwiched between the outer layer 106 and an inner layer 108 again of polyethylene material, and heat fused to form said layered construction.

The middle layer 107 is a polymer layer, conveniently a polyamide or silicon rubber layer, onto which electrically conductive heating elements have been applied to form resistive conductive paths or areas, which emit heat when sufficient electrical current is passed through them. Various alternative techniques for producing such paths/areas are known, but for flexibility, it is preferred to form the tracks form certain materials and by certain techniques as described below:

1) Foils or layers, for example foils of aluminium, copper, iron or alloys thereof, or conductive ceramics (so called electro-ceramics) such as molybdenum disilicide or indium tin oxide (ITO), which are cut, etched or otherwise formed into conductive paths, for example by laser ablation, or etching via chemicals, light or electron beam evaporation, or are deposited in thin films for example by vapor deposition, spluttering or 3D printing;

2) Pre-formed thin strands such as wires of aluminium, copper iron, nickel, platinum, nichrome, kanthal, cupronickel, arranged in a circuitous path;

3) Printed tracks, printed by means of for example screen printing or droplet printing of conductive inks such as silver-based conductive ink, copper-based conductive ink, conductive polymer ink, and carbon-based resistive ink.

Combinations of the above techniques and materials is envisaged also.

One particularly important feature of the heating elements is that they should avoid obscuring the view of the culture volume 101. To this end, thin widths of foil tracks can be manufactured or the foil itself can be made thin enough to be substantially transparent. Thin strands or wires can also be employed. Particularly favoured is the use of ITO because that material is transparent and has the additional advantage of acting like a mirror in the infra red part of the spectrum, thereby reflecting back IR radiation emanating from the volume 101 back into the volume, and thereby reducing heat loss from the bag. ITO can be deposited a layer of 10-100 nm for example via vapour deposition onto a carrier layer 107 of optical grade polyester, to provide light transmission in excess of 90%, although layers up to 100 μm are envisaged. Thus, it will be understood that the term ‘heating element’ or ‘heating elements’ is intended to encompass discrete conductive paths, as well as thin continuous films.

FIG. 2c shows an exploded view of one example of the construction of the top wall 105. Shown more clearly is the outer layer 106 heat fused with an outer part 120′ of the manifold 120, the inner layer 108 again heat fused with an inner part 120″ of the manifold 120, and the heating element carrier layer 107. In this case the heating element is a circuitously laid single wire formed from multiple fine strands of nichrome alloy, for example 24 AWG formed from 100 strands, all carried on a polyamide carrier layer 107 with a gap of at least about 5 mm between the wire runs. The wire is not laid near the manifold 101. The layers 106,107 and 108 are pressed together and heat is applied to join them by heat fusing. In this case, the layers are joined also to the inner bottom wall 115 by further heat fusing at peripheral regions 110, to form a bag. Bare ends of the wire 109 are left exposed beyond the region 110 so that an electrical supply can be connected to generate heat. That construction allows flexibility, and does not wholly obscure the culture volume 101.

FIG. 2d shows an alternative construction of the middle layer 107. In this embodiment, the heating element 109 is formed from a 10-100 nm layer of ITO deposited via vapour deposition within a mask to provide a conducive track about 100 mm wide. The track is adhered to a polyamide carrier layer to provide a substantially transparent finished construction of the middle layer 107. Because the heating element 109 in this embodiment is substantially transparent, there is no need to have viewing gaps between elements, as shown in FIG. 2c.

The invention also includes a layered heated sheet 205 which is shown in exploded view in FIG. 3. This construction includes: an outer layer 206; a middle layer 207 and an inner layer 208, all constructed and joined in a similar manner to the layers 106, 107 and 108 respectively. In this embodiment, an aperture 220 is formed in each layer. Optionally, an adhesive layer 210 can be employed which is adhered to the underside of the inner layer 208 during manufacture and includes a peel-off backing layer 211 for keeping the adhesive clean until use. The purpose of the heated sheet is to heat the top wall of a conventional bioreactor bag 10, but at the same time allow visual inspection of the bag volume 11. The construction of a heating element 209 can be the same as for the heating elements 109 described above. The heater 205 further includes an aperture 220 in each layer to fit around the manifold area 220.

Heating of about 1-10 Watts is envisaged, to avoid condensation and, for safety a voltage of up to 48 volts is contemplated. The length, cross sectional area, and applied electrical power can all be selected to match the heat output required, and the amperage and/or voltage of the electrical supply to the heater can be modified to change the heat output of the heater element in use, to control, the temperature of the upper wall 105 to avoid or reduce condensation, for example based on the dew point of the head space G.

The invention is not to be seen as limited by the embodiments described above, but can be varied within the scope of the appended claims as is readily apparent to the person skilled in the art. For instance only the upper wall 105 has been shown to incorporate heating elements, but it would be possible to have the bottom wall 115 also include additional heating elements, manufactured in a similar manner to the heating elements described above. Since those bottom heating elements are likely to be used for heating culture liquid, not for the avoidance of condensation, then a higher heat output would be demanded, for example 50-100 Watts, which can be accommodated, for example by an increased cross sectional of heater elements. Additionally, there is less need for transparency because the bottom wall will be resting on a rocking tray or the like. Where the thickness of the elements 109 means that they are not inherently transparent, then areas free from those elements can be used as windows so that the culture volume can be observed.

ITO has been highlighted as a useful material for the heating elements, however alternative material could be used, for example: Doped compounds such as aluminium-doped zinc oxide (AZO) and indium-doped cadmium oxide; aluminium, gallium or indium-doped zinc oxide (AZO, GZO or IZO); carbon nanotube conductive coatings; graphene; thin metal films or nanowire covered with graphene; inherently conductive polymers such as polyaniline and PEDOT or PSS, amorphous indium-zinc oxide; silver nanoparticle-ITO hybrids.

Although electrical power to generate heat in the heating elements is most likely to be supplied via external conductive paths, it is possible to generate a current in the elements by means of induction via an induction coil adjacent the element(s), thereby removing the need for external conductive electrical connections.

A bioreactor in the form of a flexible bag, preferably formed from polymer sheets is described and illustrated, but any bioreactor or bioreactor shape which is within the ambit of the claims could be employed, for example a rigid cylindrical acrylic bioreactor could be used. However, it is preferred that the bioreactor is made low cost enough that it can be disposed of economically, and therefore it is desirable that the majority of its parts are manufactured from plastics sheet and/or moulded plastics parts.

1. A bioreactor having at least one wall defining an internal culture volume including a top wall which is at top of the bioreactor in use, wherein at least the top wall incorporates one or more electrically resistive heating elements.

2. A bioreactor according to claim 1, wherein the or each wall is formed substantially wholly from a material or materials which is transparent, or wherein the, or each heating element is sufficiently narrow so as not to obscure, or completely obscure, the culture volume; or wherein and/or the wall or walls which have heating elements have an area which includes heating element(s) and a further area where the element(s) are absent so that the culture volume of the bioreactor is visible at least through the element free area.

3. A bioreactor according to claim 1, wherein the bioreactor is formed substantially as a flexible bag-like bioreactor and the or each wall is formed from a polymer material incorporating said element(s) in the respective wall(s) or on the surface of said wall(s).

4. A bioreactor according to claim 3, wherein the bioreactor comprises a bottom wall and a top wall each formed from sheet materials and joined at their edges to define therebetween the culture volume in use.

5. A bioreactor according to claim 4, wherein the top wall at least is a layered construction including an outer layer, an inner layer facing the volume and a heater element carrying layer, between the outer and inner layers, all joined to form the top wall.

6. A bioreactor according to claim 5, wherein the heater element carrying layer carries the heater element(s), and the element(s) are formed by one or more of the following techniques:
a) cutting, etching, ablation or evaporation of pre-existing electrically conductive films or foils;
b) depositing of conductive material or materials by means of vapour deposition, spluttering or printing including 3D printing; and
c) preforming or shaping of conductive materials such as wires, cables, or strands.

7. A bioreactor according to claim 5, wherein said element materials are selected from the group consisting of one or more of: aluminium; copper; iron; nickel; platinum; nichrome; kanthal; cupronickel; silver-based conductive ink, copper-based conductive ink, conductive polymer ink, and carbon-based resistive ink; conductive ceramics, molybdenum disilicide or indium tin oxide (ITO aluminium-doped zinc oxide (AZO) and indium-doped cadmium oxide; aluminium, gallium or indium-doped zinc oxide (AZO, GZO or IZO); carbon nanotube conductive coatings; graphene; thin metal films or nanowire covered with graphene; inherently conductive polymers, polyaniline and PEDOT or PSS; amorphous indium-zinc oxide; and silver nanoparticle-ITO hybrids.

8. A layered sheet heater for bioreactors comprising, one or more layers of flexible transparent polymer sheets, one or more electrically resistive heating elements in or on at least one of the polymer sheets, a layer of adhesive on at least one of the polymer sheets, and an optional peelable backing layer over said adhesive layer, wherein either: said layered sheet heater is transparent over substantially its whole area; or wherein heating elements are opaque but formed with gaps therebetween of sufficient width so as not to obscure, or not completely obscure the majority (<50%) of light from propagating through the layers of the heater; or wherein the or each layer has a portion which is free from said heating elements, so as to provide said transparency in the element-free area.
